(19) United States
(12) Patent Application Publication
(10) Pub. No.: US 2026/0061201 A1
(43) Pub. Date: Mar. 5, 2026

(54) MULTIPOLAR CONDUCTION SYSTEM PACING LEAD WITH ATRIAL SENSING

(71) Applicant: Cardiac Pacemakers, Inc., St. Paul, MN (US)

(72) Inventors: Andrew L. DeKock, Ham Lake, MN (US); Keith L. Hermann, Minneapolis, MN (US); G. Shantanu Reddy, Minneapolis, MN (US)

(21) Appl. No.: 19/313,689

(22) Filed: Aug. 28, 2025

Related U.S. Application Data
(60) Provisional application No. 63/688,719, filed on Aug. 29, 2024.

(51) Int. Cl. A61N 1/368; A61N 1/05

(52) U.S. Cl. CPC A61N 1/3684; A61N 1/0563; A61N 1/0573

(57) ABSTRACT

An implantable lead for use with an implantable medical device (IMD) includes a tubular lead body having a proximal portion including a proximal end, a distal portion including a distal end opposite the proximal end, and an intermediate portion between the proximal portion and the distal portion. A helical electrode extends distally from the distal end of the tubular lead body. At least one atrial electrode is disposed in the intermediate portion of the tubular lead body. At least one electrical conductor extends through the tubular lead body and is mechanically and electrically coupled to the helical electrode and the at least one atrial electrode.

### CROSS REFERENCE TO RELATED APPLICATIONS

The present application claims priority to U.S. Provisional Patent Application No. 63/688,719, filed Aug. 29, 2024, the disclosure of which is incorporated herein in its entirety.

### TECHNICAL FIELD

The present disclosure relates to medical electrical leads and associated manufacturing methods and methods of use. In particular, the present disclosure relates to implantable medical electrical leads for stimulating the conduction system of the heart.

### BACKGROUND

Cardiac rhythm management systems are useful for electrically stimulating a patient's heart to treat various cardia arrhythmias. Stimulating the native conduction system of the heart, e.g., the bundle of His, the left bundle branch and/or the right bundle branch can simultaneously pace both the right and left ventricles of the heart, potentially avoiding pacing induced dyssynchrony which may occur with right ventricular apex pacing.

Currently available pacing leads include one or two electrodes positioned at the distal end. In some cases, the left bundle branch may be difficult to target or capture using these available pacing leads. After implanting, these leads may shift positions slightly with possible loss to the left bundle branch capture. It would be beneficial improve available conduction system pacing designs to allow for more electrical vector selection, to enable electrical re-positioning to recapture the left bundle branch after shifting. Moreover, it would be valuable for a physician to pace and or sense electrical signals from structures other than the left bundle branch, such as the right bundle branch, adjacent myocardium, or the surface myocardium.

### SUMMARY

Example 1 is an implantable lead for use with an implantable medical device (IMD). The implantable lead includes a tubular lead body having a proximal portion including a proximal end, a distal portion including a distal end opposite the proximal end, and an intermediate portion between the proximal portion and the distal portion. The intermediate portion includes a pre-formed bend. A helical electrode extends distally from the distal end of the tubular lead body. At least one atrial electrode is disposed in the intermediate portion of the tubular lead body. The pre-formed bend of the intermediate portion is configured to urge the at least one atrial electrode against a surface of the right atrium when the lead is implanted. A plurality of electrical conductors extend through the tubular lead body and are mechanically and electrically coupled to respective ones of the helical electrode and the at least one atrial electrode.

Example 2 is the implantable lead of Example 1, wherein the at least one atrial electrode comprises a first atrial electrode and a second atrial electrode.

Example 3 is the implantable lead of Example 2, wherein the at least one atrial electrode comprises a third atrial electrode.

Example 4 is the implantable lead of Example 1, wherein the at least one atrial electrode is located proximate an apex of the intermediate pre-formed bend.

Example 5 is the implantable lead of any of Examples 1-4, further comprising a distal pre-formed bend located in the distal portion of the tubular lead body, the distal pre-formed bend configured to bias the helical electrode against a surface of a right ventricle.

Example 6 is the implantable lead of any of Examples 1-5, further comprising a shocking coil disposed in the distal portion of the tubular lead body.

Example 7 is the implantable lead of any of Examples 1-6, further comprising a plurality of pacing/sensing electrodes disposed in the distal portion of the lead body proximal to the helical electrode.

Example 8 is the implantable lead of Example 7, wherein the plurality of pacing/sensing electrodes comprises a first pacing/sensing electrode and a second pacing/sensing electrode, the first and second pacing/sensing electrodes defining a first pair of pacing/sensing electrodes.

Example 9 is the implantable lead of Example 8, wherein the plurality of pacing/sensing electrodes includes a third pacing/sensing electrodes spaced from the first pair of pacing/sensing electrodes.

Example 10 is the implantable lead of Example 9, wherein the third pacing/sensing electrode forms a bipolar pair with the helical electrode.

Example 11 is the implantable lead of Example 7, wherein the plurality of pacing/sensing electrodes comprise a plurality of electrode segments circumferentially spaced from one another about the tubular lead body.

Example 12 is the implantable lead of Example 11, wherein the electrode segments are positioned at the same longitudinal position on the distal portion of the lead body.

Example 13 is the implantable lead of Example 12, wherein each of the electrode segments is electrically connected to a separate electrical conductor such that the electrode segments are electrically isolated from one another.

Example 14 is the implantable lead of Example 7, wherein the plurality of pacing/sensing electrodes comprise a plurality of spot electrodes spaced from one another along the distal portion of the lead body.

Example 15 is the implantable lead of Example 7, wherein the plurality of pacing/sensing electrodes comprise a first ring electrode having an outer surface, and a second spot electrode incorporated into the outer surface of the first ring electrode.

Example 16 is an implantable lead for use with an implantable medical device (IMD). The implantable lead includes a tubular lead body having a proximal portion including a proximal end, a distal portion including a distal end opposite the proximal end, and an intermediate portion between the proximal portion and the distal portion. A helical electrode extends distally from the distal end of the tubular lead body. A plurality of pacing/sensing electrodes are located in the distal portion. At least one electrical conductor extends through the tubular lead body and is mechanically and electrically coupled to the helical electrode and the plurality of pacing/sensing electrode.

Example 17 is the implantable lead of Example 16, further comprising a proximal connector at the proximal end of the lead body configured for mechanically and electrically coupling the lead to the IMD, wherein the at least one electrical conductor is mechanically and electrically coupled to the proximal connector.

Example 18 is the implantable lead of any of Examples 16 or 17, wherein the plurality of pacing/sensing electrodes are evenly spaced apart from one another.

Example 19 is the implantable lead of any of Examples 16-18, wherein the plurality of pacing/sensing electrodes comprises a first pair of pacing/sensing electrodes.

Example 20 is the implantable lead of Example 19, wherein the first pair of pacing/sensing electrodes is spaced from a third pacing/sensing electrode.

Example 21 is the implantable lead of Example 20, wherein the third pacing/sensing electrode forms a bipolar pair with the helical electrode.

Example 22 is the implantable lead of any of Examples 16-21, wherein the plurality of pacing/sensing electrodes comprise ring electrodes that are flush with an outer surface of the tubular lead body.

Example 23 is the implantable lead of any of Examples 16-22, further comprising an intermediate pre-formed bend located in the intermediate portion of the tubular lead body.

Example 24 is the implantable lead of any of Examples 16-23, further comprising a distal pre-formed bend located in the distal portion of the tubular lead body, the distal pre-formed bend configured to bias the plurality of pacing/sensing electrodes towards a surface of a right ventricle.

Example 25 is the implantable lead of any of Examples 16-24, wherein the plurality of pacing/sensing electrodes comprise at least one segmented electrode.

Example 26 is the implantable lead of Example 25, wherein the at least one segmented electrode comprises a first and second electrode positioned at the same longitudinal position along the distal portion of the tubular lead body.

Example 27 is the implantable lead of Example 26, wherein the at least one segmented electrode comprises a third and fourth electrode positioned at the same longitudinal position as the first and second electrode.

Example 28 is the implantable lead of any of Examples 16-27, wherein the plurality of pacing/sensing electrodes comprise spot electrodes.

Example 29 is the implantable lead of Example 28, wherein the spot electrodes are rectangular, circular, oval, polygonal, square, or ring shaped.

Example 30 is the implantable lead of any of Examples 16-29, wherein the plurality of pacing/sensing electrodes comprise a first ring electrode having an outer surface, and a second spot electrode incorporated into the outer surface of the first ring electrode.

Example 31 is an implantable lead for use with an implantable medical device (IMD). The implantable lead includes a tubular lead body having a proximal portion including a proximal end, a distal portion including a distal end opposite the proximal end, and an intermediate portion between the proximal portion and the distal portion. The intermediate portion includes a pre-formed bend. At least one atrial electrode is disposed in the intermediate portion of the tubular lead body. The pre-formed bend of the intermediate portion is configured to urge the at least one atrial electrode against a surface of the right atrium when the lead is implanted. At least one pacing/sensing electrode is located in the distal portion. A helical electrode extends distally from the distal end of the tubular lead body. A plurality of electrical conductors extend through the tubular lead body and are mechanically and electrically coupled to respective ones of the helical electrode, the at least one atrial electrode, and the at least one pacing/sensing electrode.

Example 32 is the implantable lead of Example 31, wherein the at least one atrial electrode comprises a first atrial electrode and a second atrial electrode.

Example 33 is the implantable lead of Example 32, wherein the at least one atrial electrode comprises a third atrial electrode.

Example 34 is the implantable lead of Example 31, wherein the at least one atrial electrode is located proximate an apex of the intermediate pre-formed bend.

Example 35 is the implantable lead of any of Examples 31-34, further comprising a distal pre-formed bend located in the distal portion of the tubular lead body, the distal pre-formed bend configured to bias the helical electrode and the at least one pacing/sensing against a surface of a right ventricle.

Example 36 is the implantable lead of any of Examples 31-35, further comprising a shocking coil disposed in the distal portion of the tubular lead body.

Example 37 is the implantable lead of any of Examples 31-36, wherein the at least one pacing/sensing electrode comprises a plurality of pacing/sensing electrodes.

Example 38 is the implantable lead of Example 37, wherein the plurality of pacing/sensing electrodes comprises a first pacing/sensing electrode and a second pacing/sensing electrode, the first and second pacing/sensing electrodes defining a first pair of pacing/sensing electrodes.

Example 39 is the implantable lead of Example 38, wherein the plurality of pacing/sensing electrodes includes a third pacing/sensing electrodes spaced from the first pair of pacing/sensing electrodes.

Example 40 is the implantable lead of Example 39, wherein the third pacing/sensing electrode forms a bipolar pair with the helical electrode.

Example 41 is the implantable lead of Example 37, wherein the plurality of pacing/sensing electrodes comprise a plurality of electrode segments circumferentially spaced from one another about the tubular lead body.

Example 42 is the implantable lead of Example 41, wherein the electrode segments are positioned at the same longitudinal position on the distal portion of the lead body.

Example 43 is the implantable lead of Example 42, wherein each of the electrode segments is electrically connected to a separate electrical conductor such that the electrode segments are electrically isolated from one another.

Example 44 is the implantable lead of Example 37, wherein the plurality of pacing/sensing electrodes comprise a plurality of spot electrodes spaced from one another along the distal portion of the lead body.

Example 45 is the implantable lead of Example 37, wherein the plurality of spot electrodes are rectangular, circular, oval, polygonal, square, or ring shaped.

Example 46 is the implantable lead of Example 37, wherein the plurality of pacing/sensing electrodes comprise a first ring electrode having an outer surface, and a second spot electrode incorporated into the outer surface of the first ring electrode.

Example 47 is the implantable lead of any of Examples 31-46, further comprising a proximal connector at the proximal end of the lead body configured for mechanically and electrically coupling the lead to the IMD, wherein the plurality of electrical conductors is mechanically and electrically coupled to the proximal connector.

Example 48 is the implantable lead of Example 31, wherein the at least one atrial electrode or the at least one pacing/sensing electrode comprises a ring electrode that is flush with an outer surface of the tubular lead body.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram of a conduction system pacing (CSP) system, according to some embodiments of this disclosure.

FIG. 2 illustrates a first embodiment of a lead for use in the system of FIG. 1, in accordance with embodiments of the subject matter of the disclosure.

FIGS. 3A and 3B illustrate cross-sectional views along line 3-3 of FIG. 2, in accordance with embodiment of the subject matter of the disclosure.

FIG. 4 illustrates a second embodiment of a lead for use in the system of FIG. 1, in accordance with embodiments of the subject matter of the disclosure.

FIG. 5 illustrates a third embodiment of a lead illustrates a lead for use in the system of FIG. 1, in accordance with embodiments of the subject matter of the disclosure.

FIG. 6 illustrates a fourth embodiment of a lead illustrates a lead for use in the system of FIG. 1, in accordance with embodiments of the subject matter of the disclosure.

FIG. 7 illustrates a first arrangement of electrodes of the lead of FIG. 6, in accordance with an embodiment of the present disclosure.

FIG. 8 illustrates a second arrangement of electrodes of the lead of FIG. 6, in accordance with an embodiment of the present disclosure.

FIGS. 9A and 9B illustrate an arrangement for electrodes for use with a lead, in accordance with an embodiment of the present disclosure.

FIG. 10 illustrates a lead having a plurality of various electrodes for use in the system of FIG. 1, in accordance with an embodiment of the present disclosure.

FIG. 11 illustrates various arrangements of electrodes for use with a lead, in accordance with an embodiment of the present disclosure.

FIG. 12 is a perspective view of a ring electrode having an additional electrode formed therein, in accordance with an embodiment of the present disclosure.

FIG. 13 is a cross-sectional view of the ring electrode of FIG. 12 along line 13-13, in accordance with an embodiment of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

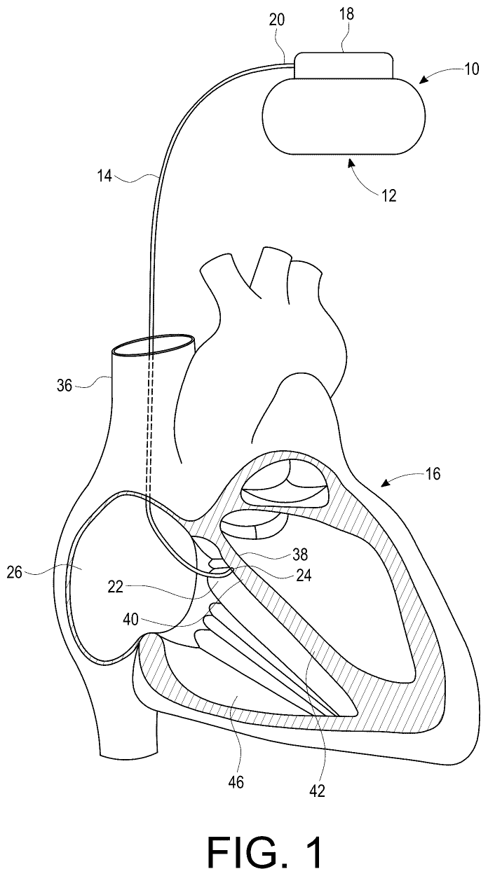

FIG. 1 is a schematic diagram of a conduction system pacing (CSP) system, according to some embodiments of this disclosure. FIG. 1 illustrates a CSP system 10 including an implantable medical device (IMD) such as an implantable pulse generator 12 and a lead 14. The lead 14 is implanted in a heart 16. The implantable pulse generator 12 can include circuitry for sensing bioelectrical signals and/or delivering electrical stimulation via the lead 14. The implantable pulse generator 12 can include a lead interface 18 (e.g., a header). The lead 14 can include a proximal end 20, a distal end 22, and a fixation element 24 disposed at the distal end 22.

The lead 14 can further include a proximal connector having one or more electrical contacts (not shown) at the proximal end 20, one or more electrical elements (e.g. ring electrodes) at the distal end 22, and one or more electrical conductors (e.g., one or more coils or one or more cable conductors) (not shown) extending within one or more lumens extending within the lead 14 from the electrical contacts to the electrical elements. The lead interface 18 can connect the pulse generator 12 to the electrical contacts at the proximal end 20 of the lead 14 to electrically connect the pulse generator 12 to the electrical elements.

As shown in FIG. 1, the lead 14 is implanted in a right ventricle 46, at a ventricular septum 42, proximate a left bundle branch 38 and/or right bundle branch 40 of the specialized conduction system. The lead 14 operates to convey electrical signals between the target nerve(s), e.g., the left bundle branch 38 and/or the right bundle branch 40 and the implantable pulse generator 12. In some embodiments, the lead 14 can enter the vascular system through a vascular entry site (not shown) formed in a wall of the left subclavian vein (not shown), extend through the left brachiocephalic vein (not shown) and the superior vena cava 36, to the right ventricle 46. Other suitable vascular access sites may be used in various other embodiments.

The fixation element 24 can fix the lead 14 to cardiac tissue, such as the area of tissue by which the left bundle branch 38 and/or the right bundle branch 40 can be directly stimulated. In some embodiments, the fixation element 24 can be electrically coupled to the implantable pulse generator 12 by, for example, one of the electrical conductors, such as a coil, extending to the proximal end 20 of the lead 14 for interfacing with the lead interface 18. As such, the fixation element 24 can mechanically and electrically couple the lead 14 to the tissue and facilitate the transmission of electrical energy from the conduction system in a sensing mode and to conduction system in a stimulation mode. In some embodiments, the fixation element 24 is a fixed fixation element, such as helix fixed to the lead 14. Such a fixation element 24 can be deployed by rotating the lead 14 itself to implant the fixation element 24 into the tissue. The use of the active fixation element for the fixation element 24 may allow for precise placement of the lead 14. The use of the active fixation element for the fixation element 24 may also provide for mapping capability because the user need not be concerned with accidental entanglement of the helix in the tissue.

While FIG. 1 only shows a single lead connected to the implantable pulse generator 12 and implanted for cardiac stimulation, various other embodiments can have an alternative lead and/or one or more additional leads for sensing bioelectrical activity and/or stimulating other areas of the heart 16.

In some embodiments, as will be discussed in greater detail herein, the CSP system 10 can be capable of both pacing and defibrillation therapies. In such embodiments, the lead 14 can also include one or more high voltage defibrillation electrodes (not shown in FIG. 1) for delivering defibrillation shocks capable of terminating ventricular fibrillation.

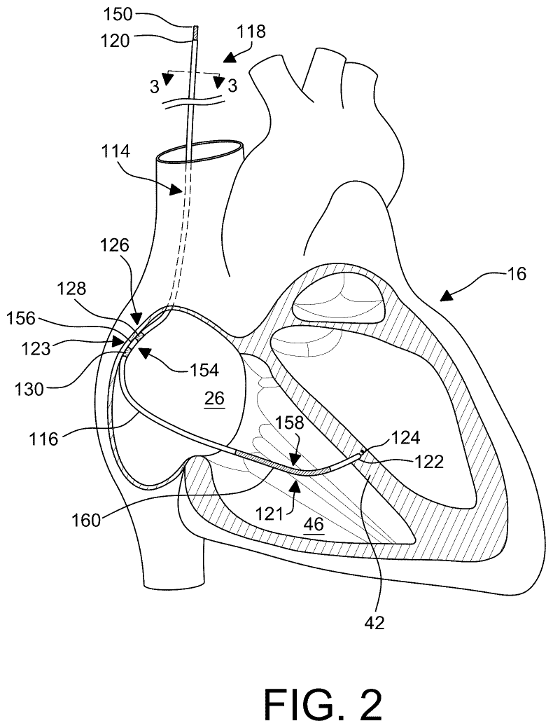

FIG. 2 illustrates a first embodiment of an implantable lead for use in the system of FIG. 1, in accordance with embodiments of the subject matter of the disclosure. The implantable lead 114 can be used in a conduction system capable of delivering energy to a target location in a heart 16 as well as sensing or pacing other locations in the heart 16. The implantable lead 114 includes a tubular lead body 116 having a proximal portion 118 including a proximal end 120. The tubular body includes a distal portion 121 including a distal end 122 opposite the proximal end 120. An intermediate portion 123 is located between the proximal portion 118 and the distal portion 121. The tubular lead body 116 has an outer diameter that reduces in diameter towards the distal end 122. This allows the distal portion 121 of the lead body 116 to be partially inserted into heart tissue, such as a ventricular septum 42, to allow stimulation or monitoring of a left bundle branch 38 and/or right bundle branch 40.

The tubular lead body 116 can be formed of an electrically insulative material or a conductive material coated with an insulative material. Insulative materials may include a polymeric material or a ceramic material. Polymeric materials may include polyetheretherketone, epoxy, polyurethane, or parylene, for example. Ceramic materials may be deposited, fired, molded, and/or machined.

A fixation element in the form of a helical electrode 124 extends distally from the distal end 122 of the tubular lead body 116. The length of the helical electrode 124 can be selected according to the particular clinical needs. In embodiments, the helical electrode 124 extends from the distal end 122 of the tubular lead body 116 by a distance in the range of from about 0.1 millimeters to about 5.0 millimeters. In some embodiments, the helical electrode 124 extends from the distal end 122 of the tubular lead body 116 by a distance in the range of from about 0.5 mm to about 2.5 mm. In still other embodiments, a longer helical electrode 124 may be utilized. By way of example only, in unique cases a helical electrode 124 having a length of up to about 16 millimeters could be employed to enable full reach across the septum 42 to reach the left bundle branch 38 (see FIG. 2) without the need for the lead tip to tunnel into the septal tissue. The helical electrode 124 has a sharp tip to allow for insertion into tissue and one or more turns to screw into the tissue. In some embodiments, a stylet can be extended through the lead body 116 to engage the helical electrode 124 such that rotation of the stylet rotates the helical electrode 124 to drive the helical electrode 124 into tissue. In other embodiments, rotation of the lead body 116 drives the helical electrode 124 into tissue. In some embodiments, the helical electrode 124 is active and can be used as a pacing or sensing electrode. In other embodiments, the helical electrode 124 is not active and is simply intended to anchor the lead 114 at a desired location.

As shown, the implantable lead 114 includes an atrial electrode pair 126 including a first atrial electrode 128 and a second atrial electrode 130 disposed in the intermediate portion 123 of the tubular lead body 116. The atrial electrode pair 126 is configured to be located in the right atrium 26 of a heart 16 when the lead 114 is implanted and, in embodiments, is configured for bipolar sensing of atrial activity. This can facilitate detection of atrial high rate and be incorporated into discrimination algorithms allowing a reduction in the rate of inappropriate or unnecessary shocks delivered. The atrial electrode 126 can include a plurality of electrodes for sensing atrial tissue.

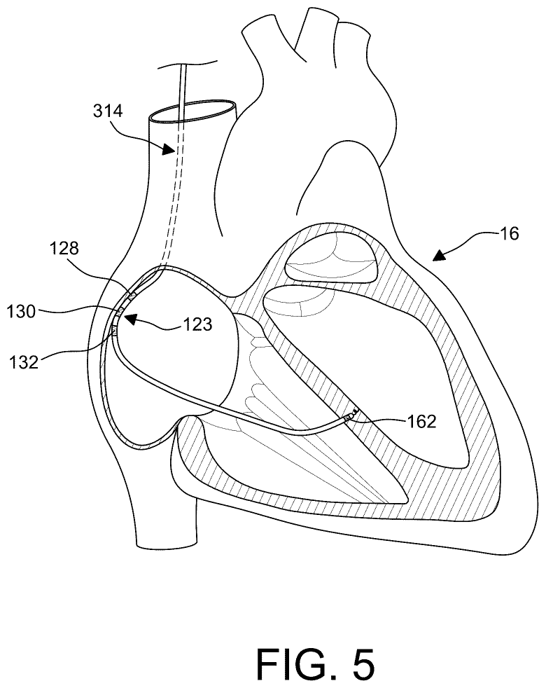

In various embodiments, the implantable lead 114 may include atrial sense electrode configurations that are different than that shown in FIG. 2. For example, FIG. 5 illustrates an embodiment including a first atrial electrode 128, a second atrial electrode 130, and a third atrial electrode 132. While three atrial electrodes are illustrated in FIG. 5, more electrodes can be located in the intermediate portion 123 as desired. In other embodiments, only a single atrial sense electrode, e.g., the atrial electrode 128 or 130, may be included.

In some embodiments, each of the atrial electrodes 128, 130 is a ring electrode. The ring electrode can include a substantially ring-shaped or cylindrical conductive material that circumscribes a portion of the lead body 116. In some embodiments, the ring electrode can be located in a groove or recess such that an outer surfaces of the atrial electrodes 128, 130 are substantially flush with an outer surface of the tubular lead body 116. In some embodiments, the outer surface of the ring electrodes is below an outer surface of the tubular lead body 116. In other embodiments, the outer surface of the ring electrodes may extend nominally above the outer surface of the tubular lead body 116.

The helical electrode 124 and the atrial electrodes 128, 130 are each constructed from an electrically conductive material. In some embodiments, the helical electrode and the at least one atrial electrode include MP35N, Elgiloy, MP35N LT, platinum alloys, stainless steel alloys, palladium alloys, and titanium. In some embodiments, the helical electrode 124 and the atrial electrodes 128, 130 include any of the foregoing conductive material plated or deposited by powdered metallurgy over a ceramic material or a polymer material. In some embodiments, the helical electrode 124 and the atrial electrodes 128, 130 include a conductive material that is radiopaque, such as tungsten, platinum alloys, palladium alloys or iridium alloys, for example.

As shown, the tubular lead body 116 is biased into an implanted arrangement to direct the atrial electrodes 128, 130 against a desired tissue surface in the right atrium 26 and to direct the helical electrode 124 towards a desired tissue surface in the right ventricle 46, such as the ventricular septum 42. In order to create the desired biased shape of the lead body 116, an intermediate pre-formed bend 154 is located in the intermediate portion 123 of the tubular lead body 116. The intermediate pre-formed bend 154 is configured to bias the atrial electrodes 128, 130 against a surface of a right atrium 26. The intermediate pre-formed bend 154 includes an apex 156, and the atrial electrodes 128, 130 are located proximate the apex.

In the illustrated embodiment, a shocking coil 160 is disposed in the distal portion 121 of the tubular lead body 116. As will be appreciated, when present, the shocking coil 160 can be used to delivery high voltage cardioversion shocks for terminating a potentially fatal arrhythmia (e.g., ventricular fibrillation). In embodiments, the tubular lead body 116 may also include a distal pre-formed bend 158 located in the distal portion 121 of the tubular lead body 116. When present, the distal pre-formed bend 158 is configured to bias the helical electrode 124 towards a surface of a right ventricle 42.

As illustrated in FIG. 2, the intermediate pre-formed bend 154 causes the lead body 116 to bend towards a surface of the right atrium 26 at an exterior of the heart 16 when the lead 114 is in its implanted state. The pre-formed bend can be exaggerated or reduced as desired to allow for placement of the atrial electrodes 128, 130 at a desired location within the right atrium 26 and placement of the shocking coil 160 or helical electrode 124 at a desired location within the right ventricle 46, for example at the ventricular septum 42.

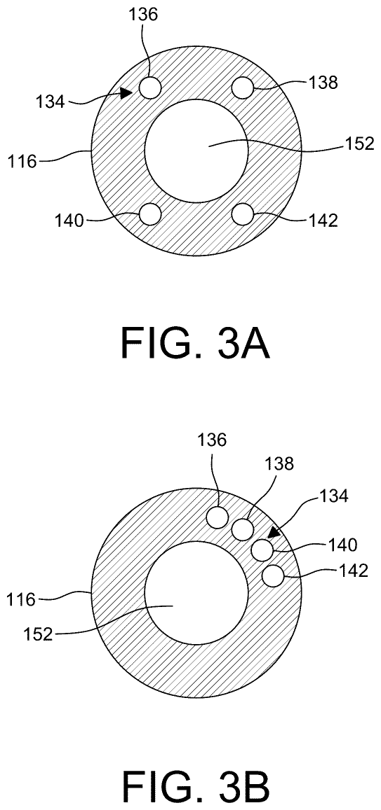

FIGS. 3A and 3B are schematic cross-sectional views of the lead 114 taken along line 3-3 of FIG. 2, in accordance with embodiment of the subject matter of the disclosure. As shown, in order to connect the helical electrode 124 and the at least one atrial electrode 126 to the IMD, for example an implantable pulse generator 12, at least one electrical conductor 134 extends through the tubular lead body 116 and is mechanically and electrically coupled to the helical electrode 124 and the at least one atrial electrode 126. In some embodiments, a single conductor connects to the helical electrode 124 and the at least one atrial electrode 126. In other embodiments, each electrode is electrically coupled to the IMD by a dedicated conductor, such that each electrode can transmit a signal or receive energy for application to tissue independently of each other. For example, as shown in FIGS. 3A and 3B, a first electrical conductor 136 is mechanically and electrically connected to the helical electrode 124, a second electrical conductor 138 is mechanically and electrically connected to a first atrial electrode 128, a third electrical conductor 140 is mechanically and electrically connected to a second atrial electrode 130, and a fourth electrical conductor 142 is mechanically and electrically connected to a third atrial electrode 132.

The at least one electrical conductor 134 extends proximally from a respective electrode through the lead body 116 to a proximal connector 150 located at the proximal end 120 of the lead body 116. The at least one electrical conductor 134 is mechanically and electrically coupled to the proximal connector 150. The proximal connector 150 is configured for mechanically and electrically coupling the lead to the IMD. In some embodiments, the proximal connector 150 is a pin connector. In some embodiments, the proximal connector 150 is an IS-4, four-pole connector. In some embodiments, the proximal connector 150 is configured to support connection to more than four independently controlled electrodes.

As shown in FIG. 3A, in embodiments where the at least one electrical conductor 134 includes a plurality of conductors 136, 138, 140, 142, the plurality of conductors 136, 138, 140, 142 can be arranged uniformly around a lumen 152 of the lead body 116. FIG. 3B illustrates a second arrangement for the plurality of electrical conductors 136, 138, 140, 142. As illustrated in FIG. 3B, the conductors 136, 138, 140, 142 can be located substantially adjacent one another along one portion of the lead body 116.

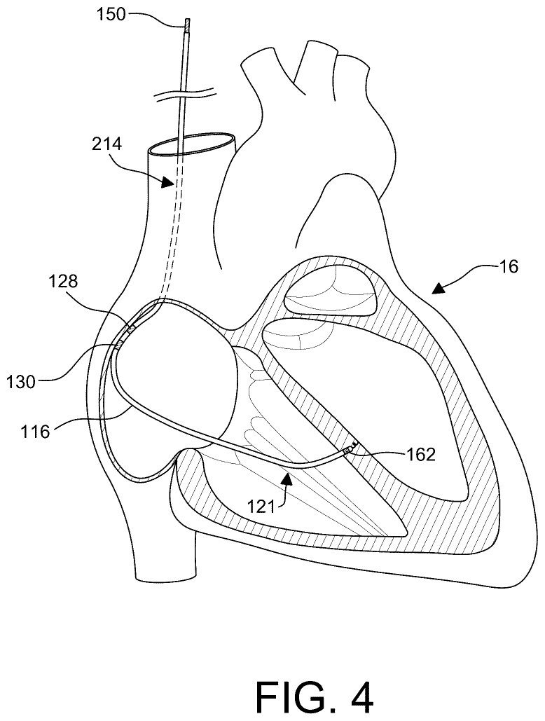

FIG. 4 illustrates a second embodiment of a lead 214 for use in the system of FIG. 1, in accordance with embodiments of the subject matter of the disclosure. The lead 214 of FIG. 4 is similar to the lead 114 of FIG. 2 and common elements are identified the same. The lead 214 of FIG. 4 varies from the lead 114 of FIG. 2 in that instead of a shocking coil 160 in the distal portion 121, the lead 214 includes at least one sensing or shocking electrode 162 disposed in the distal portion 121 of the lead body. The at least one sensing or shocking electrode 162 is located proximal of the helical electrode 124. The at least one sensing or shocking electrode 162 can be positioned such that it is capable of capturing signals from the left bundle branch 38 or the right bundle branch 40, or to deliver energy to the left bundle branch 38 or the right bundle branch 40. The at least one sensing or shocking electrode 162 is coupled to the IMD via a conductor that extends proximally through the lead body 116 to the connector 150.

FIG. 5 illustrates a third embodiment of a lead 314 for use in the system of FIG. 1, in accordance with embodiments of the subject matter of the disclosure. The lead 314 of FIG. 5 is similar to the lead 114 of FIG. 2 and common elements are identified the same. The lead 314 of FIG. 5 varies from the lead 114 of FIG. 2 in that the at least one atrial electrode 126 comprises a first atrial electrode 128, second atrial electrode 130, and third atrial electrode 132. Each of the electrodes are coupled to the IMD via a conductor that extends proximally through the lead body 314 to the connector 150. In one embodiment, each of the first 128, second 130, and third 132 atrial electrodes are electrically coupled to the same conductor. In another embodiment, each of the first 128, second 130, and third 132 atrial electrodes have their own conductor such that the first 128, second 130, and third 132 atrial electrodes can be operated independently.

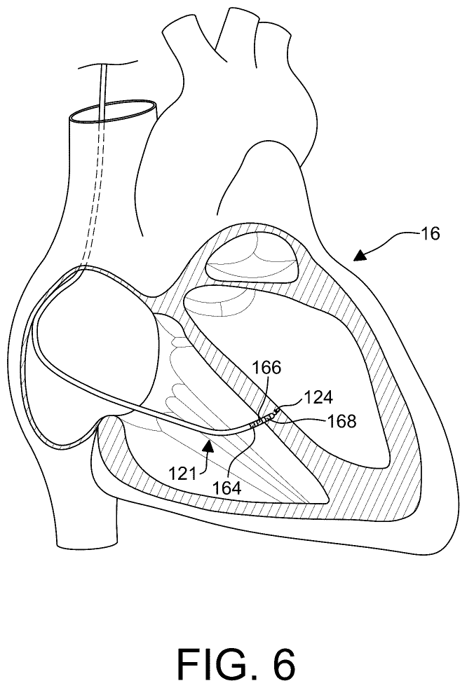

FIG. 6 illustrates a fourth embodiment of a lead 414 for use in the system of FIG. 1, in accordance with embodiments of the subject matter of the disclosure. As with previous embodiments, the implantable lead 414 includes a tubular lead body 116 having a proximal portion 118 including a proximal end 120, a distal portion 121 including a distal end 122 opposite the proximal end 120, and an intermediate portion 123 between the proximal portion 118 and the distal portion 121. A helical electrode 124 extends distally from the distal end 122 of the tubular lead body 116. In this embodiment, a plurality of sensing or pacing electrodes 164, 166, 168 are located in the distal portion 121. The plurality of sensing or pacing electrodes 164, 166, 168 comprise ring electrodes that are flush with an outer surface of the tubular lead body 116. The plurality of sensing or pacing electrodes 164, 166, 168 are configured to receive signals from ventricular tissue or apply energy to the tissue. While not illustrated, the embodiment of FIG. 6 can include one or more atrial electrodes as discussed above.

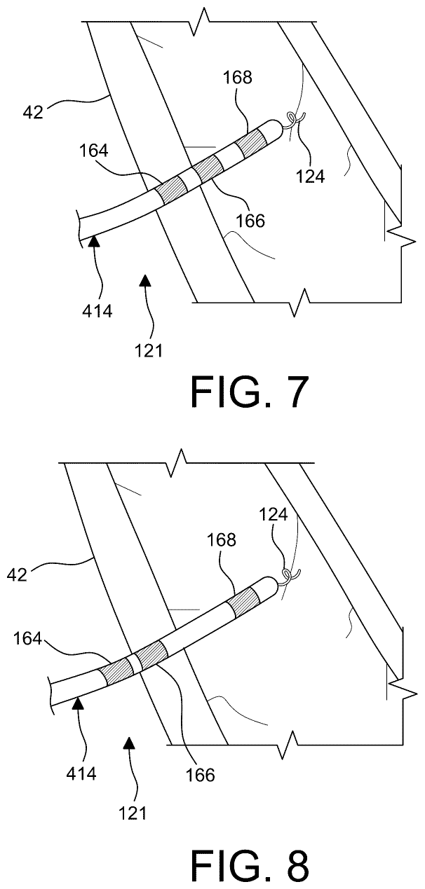

FIG. 7 illustrates a first arrangement of the plurality of sensing or pacing electrodes 164, 166, 168 of the lead 414 of FIG. 6, in accordance with an embodiment of the present disclosure. As illustrated in FIG. 7, the plurality of sensing or pacing electrodes 164, 166, 168 are evenly spaced apart from one another. The plurality of sensing or pacing electrodes 164, 166, 168 include an even spacing between the first sensing or pacing electrode 164 and the second sensing or pacing electrode 166, between the second sensing or pacing electrode 166 and the third sensing or pacing electrode 168, and between the third sensing or pacing electrode 168 and the helical electrode 124. The plurality of sensing or pacing electrodes 164, 166, 168 are arranged along the distal portion 121 of the tubular lead body 116 such that they can be placed adjacent or into a target body tissue, for example a ventricular septum 42.

FIG. 8 illustrates a second arrangement of a plurality of sensing or pacing electrodes 164, 166, 168 of the lead 414 of FIG. 6, in accordance with an embodiment of the present disclosure. As illustrated in FIG. 8, the plurality of sensing or pacing electrodes 164, 166, 168 are arranged along the distal portion 121 of the tubular lead body 116 non-uniformly. A first pair of sensing or pacing electrodes, comprising a first sensing or pacing electrode 164 and a second sensing or pacing electrode 166, are located adjacent one another and are spaced from a third sensing or pacing electrode 168. The first pair can be placed far enough from the third sensing or pacing electrode 168 such that the first sensing or pacing electrode 164 can reside outside of the tissue while the second sensing or pacing electrode 166 is located within the tissue. In one embodiment, the first pair of sensing or pacing electrodes form a bipolar pair and the third sensing or pacing electrode 168 forms a bipolar pair with the helical electrode 124.

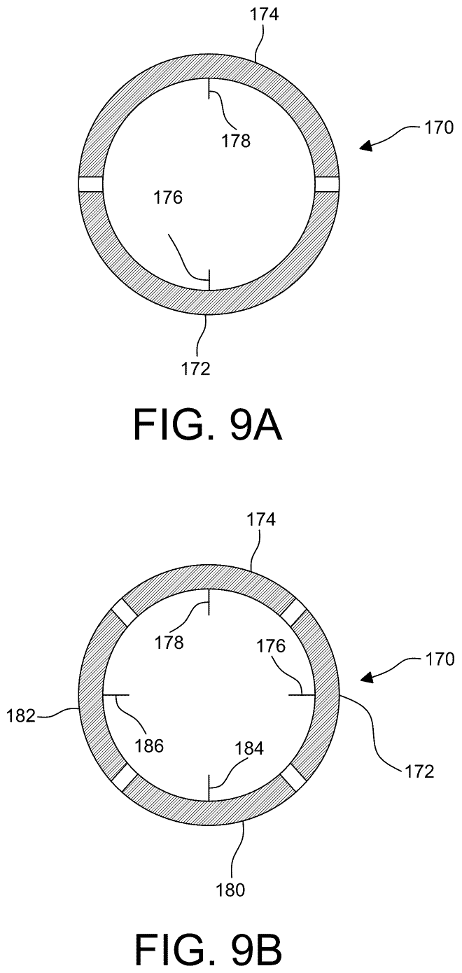

FIGS. 9A and 9B illustrate an arrangement for at least one atrial electrode or sensing or pacing electrodes for use with a lead, in accordance with an embodiment of the present disclosure. In one embodiment, the at least one atrial electrode 126 or plurality of sensing or pacing electrodes 164, 166, 168 comprise at least one segmented electrode 170. A segmented electrode 170 comprises a plurality of electrodes that share a common position longitudinally along the tubular lead body 116. A segmented electrode 170 can take the form of a ring electrode that has been separated into two or more segments, each segment being connected to an individual conductor to allow for separate operation of each segment. While FIGS. 9A and 9B illustrate the at least one segmented electrode as having a circular cross-section, in some embodiments the at least one segmented electrode includes an oval, square, rectangular, or polygonal cross-section.

As illustrated in FIG. 9A, the at least one segmented electrode 170 comprises a first electrode segment 172 and second electrode segment 174, the first and second electrode are positioned at the same longitudinal position along the distal portion 121 of the tubular lead body 116. A first conductor 176 is mechanically and electrically connected to the first electrode segment 172 of the segmented electrode 170 and a second conductor 178 is mechanically and electrically connected to the second electrode segment 174 of the segmented electrode 170. In operation, the first and second electrode can operate together or independently. The first electrode segment 172 and the second electrode segment 174 can be operated independently, to receive signals from different locations of tissue, or as a pair, for example a bipolar pair during the delivery of energy.

As illustrated in FIG. 9B, the at least one segmented electrode 170 comprises a first electrode segment 174, a second electrode segment 172, a third electrode segment 180, and a fourth electrode segment 182 positioned at the same longitudinal position along a portion of the tubular lead body 116, for example the distal portion 121. A first conductor 172 is mechanically and electrically connected to the first electrode segment 174 of the segmented electrode 170, a second conductor 178 is mechanically and electrically connected to the second electrode segment 174 of the segmented electrode 170, a third conductor 184 is mechanically and electrically connected to the third electrode segment 180 of the segmented electrode 170, and a fourth conductor 186 is mechanically and electrically connected to the fourth electrode segment 182 of the segmented electrode 170. In operation, the first 172, second 174, third 180, and fourth 182 electrode can operate together or independently. Any two of the first 172, second 174, third 180, and fourth 182 electrodes can be operated as a first pair, for example a bipolar pair during the delivery of energy, and the second two can be operated as a second pair.

While FIG. 9A illustrates a segmented electrode 170 as having two independent electrodes and FIG. 9B illustrates a segmented electrode 170 having four independent electrodes, the at least one segmented electrode 170 may include any number of electrodes greater than two. In some embodiments, the at least one segmented electrode 170 may include up to 10 electrodes. In some embodiments, the at least one segmented electrode 170 may include up to 20 electrodes. In some embodiments, the plurality of the electrodes forming the at least one segmented electrode 170 can each be wired independently to allow for independent operation of each of the plurality of electrodes. In some embodiments, a first set of the plurality of electrodes forming the at least one segmented electrode 170 can be wired to a first common conductor to allow for simultaneous operation of the first set, and a second set of the plurality of electrodes forming the at least one segmented electrode can be wired to a second common conductor to allow for simultaneous operation of the second set.

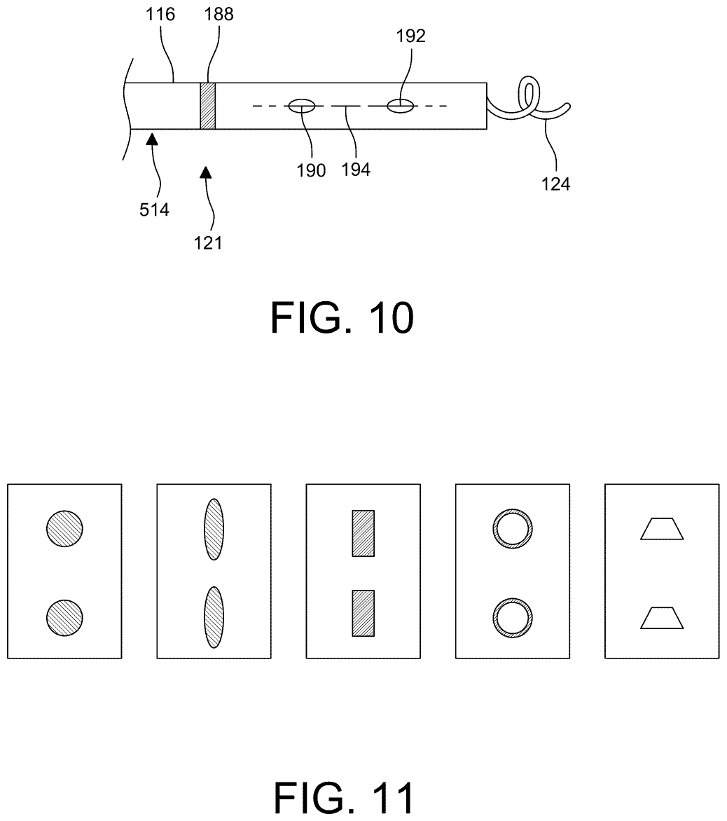

FIG. 10 illustrates a distal portion of the lead 514 having a plurality of various sensing or pacing electrodes for use in the system of FIG. 1, in accordance with an embodiment of the present disclosure. The distal portion 121 of the tubular body 116 includes a first sensing or pacing electrode 188 in the form of a ring electrode. The ring electrode is configured as a solid electrode that substantially circumscribes the entire outer surface of the tubular body at a location along the tubular lead body 116.

A second 190 and third 192 sensing or pacing electrode both take the form of a spot electrode. In some embodiments, the at least one atrial electrode 126 takes the form of a spot electrode. Spot electrodes include an active portion that spans only a portion of the outer surface of the tubular lead body 116. In some embodiments, the spot electrodes 190, 192 are arranged on the outer surface of the distal portion 121 at the same axial location, that is, along a line 194 that is parallel to a longitudinal axis of the tubular lead body 116 as illustrated in FIG. 10. In other embodiments, multiple spot electrodes can be arranged at different axial locations on the outer surface. For example, one of the spot electrodes can be located on the bottom of the tubular lead body 116, and the other spot electrode can be located substantially opposite on the top of the tubular lead body 116. In some embodiments, multiple spot electrodes can share a common longitudinal location along the length of the tubular body 116. In other embodiments, multiple spot electrodes are positioned at various longitudinal locations along the length of the tubular body as illustrated in FIG. 10. The arrangement illustrated in FIG. 10 can be used with the lead having one or more atrial electrodes as described above.

FIG. 11 illustrates various arrangements of spot electrodes for use with a lead, in accordance with an embodiment of the present disclosure. FIG. 11 illustrates pairs of spot electrodes having various shapes. Spot electrodes can be formed to have a circular, oval, rectangular (square), polygonal, or ring shape. The spot electrodes can be completely solid across the active surface or can include just an active outer perimeter of a defined shape, such as a ring.

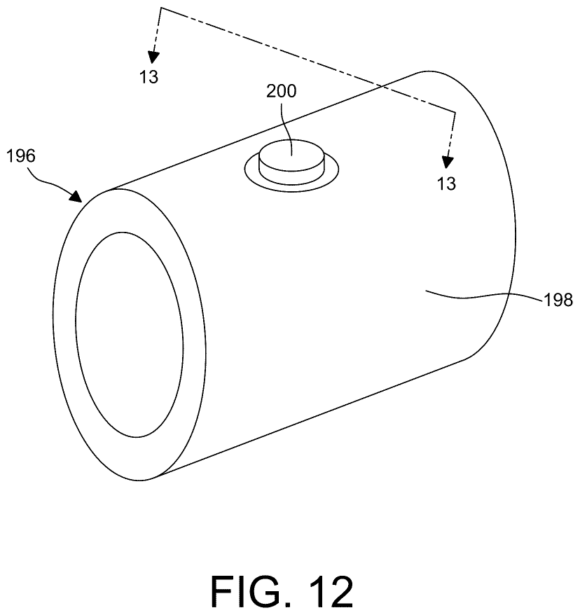

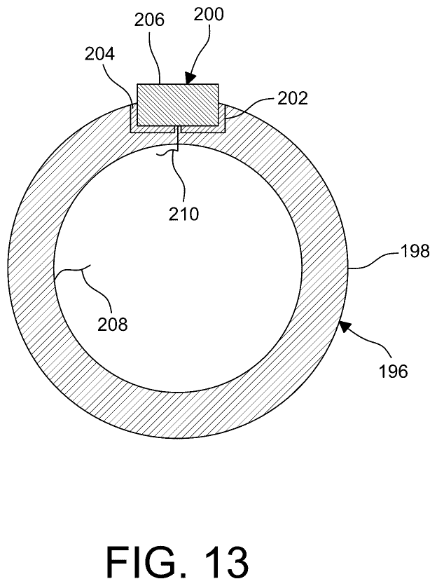

FIG. 12 is a perspective view of a ring electrode 196 having an additional electrode formed therein for use with a lead, in accordance with an embodiment of the present disclosure. The ring electrode 196 can be used as an atrial electrode, or a sensing or pacing electrode. The configuration illustrated in FIG. 12 allows for optimized sensing precision, for example for EKG timing. As illustrated in FIG. 12, the ring electrode 196 includes an outer surface 198. A spot electrode 200 is incorporated into the outer surface 198 of the ring electrode 196. The spot electrode 200 can have an active surface that is flush with the outer surface 198 of the ring electrode 196, above the outer surface 198 of the ring electrode 196, or below the outer surface 198 of the ring electrode 196. In one embodiment, as illustrated in FIG. 13, the spot electrode 200 is received in a recess 202 in the ring electrode 196. In other embodiments, the spot electrode 200 is mounted on the outer surface 198 of the ring electrode 196, separated by a layer of electrically insulated material.

FIG. 13 is a cross-sectional view of the ring electrode of FIG. 12 along line 13-13 illustrating the spot electrode 200 positioned within a recess 202 of the ring electrode 196. The spot electrode 200 is electrically insulated from the ring electrode 196 by a layer of electrically insulative material 204. The active surface 206 of the spot electrode 200 is elevated above the outer surface 198 of the ring electrode 196. A first conductor 208 is electrically and mechanically connected to the ring electrode 196 and a second conductor 210 is electrically and mechanically connected to the spot electrode 200. The first conductor 208 and second conductor 210 allow for independent operation of the ring electrode 196 and the spot electrode 200 and extend proximally through the tubular lead body 116 and connect to a proximal connector 150.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. For example, any embodiment can include one or more atrial electrodes, one or more pacing or sensing electrodes, and one or more shocking coil. Accordingly, the scope of the present disclosure is intended to embrace all such alternatives, modifications, and variations as fall within the scope of the claims, together with all equivalents thereof.

We claim:

1. An implantable lead for use with an implantable medical device (IMD), the implantable lead comprising:
a tubular lead body having a proximal portion including a proximal end, a distal portion including a distal end opposite the proximal end, and an intermediate portion between the proximal portion and the distal portion, the intermediate portion having a pre-formed bend;
a helical electrode extending distally from the distal end of the tubular lead body;
at least one atrial electrode disposed in the intermediate portion of the tubular lead body, wherein the pre-formed bend of the intermediate portion is configured to urge the at least one atrial electrode against a surface of the right atrium when the lead is implanted; and
a plurality of electrical conductors extending through the tubular lead body and mechanically and electrically coupled to respective ones of the helical electrode and the at least one atrial electrode.

2. The implantable lead of claim 1, wherein the at least one atrial electrode comprises a first atrial electrode and a second atrial electrode.

3. The implantable lead of claim 2, wherein the at least one atrial electrode comprises a third atrial electrode.

4. The implantable lead of claim 1, wherein the at least one atrial electrode is located proximate an apex of the intermediate pre-formed bend.

5. The implantable lead of claim 1, further comprising a distal pre-formed bend located in the distal portion of the tubular lead body, the distal pre-formed bend configured to bias the helical electrode against a surface of a right ventricle.

6. The implantable lead of claim 1, further comprising a shocking coil disposed in the distal portion of the tubular lead body.

7. The implantable lead of claim 1, further comprising a plurality of pacing/sensing electrodes disposed in the distal portion of the lead body proximal to the helical electrode.

8. The implantable lead of claim 7, wherein the plurality of pacing/sensing electrodes comprises a first pacing/sensing electrode and a second pacing/sensing electrode, the first and second pacing/sensing electrodes defining a first pair of pacing/sensing electrodes.

9. The implantable lead of claim 8, wherein the plurality of pacing/sensing electrodes includes a third pacing/sensing electrodes spaced from the first pair of pacing/sensing electrodes.

10. The implantable lead of claim 9, wherein the third pacing/sensing electrode forms a bipolar pair with the helical electrode.

11. The implantable lead of claim 7, wherein the plurality of pacing/sensing electrodes comprise a plurality of electrode segments circumferentially spaced from one another about the tubular lead body.

12. The implantable lead of claim 11, wherein the electrode segments are positioned at the same longitudinal position on the distal portion of the lead body.

13. The implantable lead of claim 12, wherein each of the electrode segments is electrically connected to a separate electrical conductor such that the electrode segments are electrically isolated from one another.

14. The implantable lead of claim 7, wherein the plurality of pacing/sensing electrodes comprise a plurality of spot electrodes spaced from one another along the distal portion of the lead body.

15. The implantable lead of claim 7, wherein the plurality of pacing/sensing electrodes comprise a first ring electrode having an outer surface, and a second spot electrode incorporated into the outer surface of the first ring electrode.

16. An implantable lead for use with an implantable medical device (IMD), the implantable lead comprising:
a tubular lead body having a proximal portion including a proximal end, a distal portion including a distal end opposite the proximal end, and an intermediate portion between the proximal portion and the distal portion;
a helical electrode extending distally from the distal end of the tubular lead body;
a plurality of pacing/sensing electrodes located in the distal portion; and
at least one electrical conductor extending through the tubular lead body and mechanically and electrically coupled to the helical electrode and the plurality of pacing/sensing electrode.

17. The implantable lead of claim 16, further comprising a distal pre-formed bend located in the distal portion of the tubular lead body, the distal pre-formed bend configured to bias the plurality of pacing/sensing electrodes towards a surface of a right ventricle.

18. The implantable lead of claim 16, wherein the plurality of pacing/sensing electrodes comprise at least one segmented electrode.

19. The implantable lead of claim 16, wherein the plurality of pacing/sensing electrodes comprise a first ring electrode having an outer surface, and a second spot electrode incorporated into the outer surface of the first ring electrode.

20. An implantable lead for use with an implantable medical device (IMD), the implantable lead comprising:
a tubular lead body having a proximal portion including a proximal end, a distal portion including a distal end opposite the proximal end, and an intermediate portion between the proximal portion and the distal portion, the intermediate portion having a pre-formed bend;
at least one atrial electrode disposed in the intermediate portion of the tubular lead body, wherein the pre-formed bend of the intermediate portion is configured to urge the at least one atrial electrode against a surface of the right atrium when the lead is implanted;
at least one pacing/sensing electrode located in the distal portion;
a helical electrode extending distally from the distal end of the tubular lead body; and
a plurality of electrical conductors extending through the tubular lead body and mechanically and electrically coupled to respective ones of the helical electrode, the at least one atrial electrode, and the at least one pacing/sensing electrode.
